(19) United States
(12) Patent Application Publication
(10) Pub. No.: US 2016/0106329 A1
(43) Pub. Date: Apr. 21, 2016

(54) UTILIZING DIFFERENT COLOR CHANNELS FOR RGB-IMAGE-BASED DIFFERENTIAL HEART RATE DETECTION

(71) Applicant: Microsoft Corporation, Redmond, WA (US)

(72) Inventors: Jonathan Hoof, Kenmore, WA (US); Benjamin William Walker, Seattle, WA (US); Beibei Cheng, Redmond, WA (US); Daniel Kennett, Bellevue, WA (US)

(21) Appl. No.: 14/518,522

(22) Filed: Oct. 20, 2014

(51) Int. Cl. A61B 5/024; A61B 5/00; A61B 5/0255; A61B 5/026

(52) U.S. Cl. CPC A61B 5/02427; A61B 5/0261; A61B 5/748; A61B 5/0255; A61B 5/742; A61B 5/0013; A61B 5/7278; A61B 5/486; A61B 2576/00

(57) ABSTRACT

Aspects described herein include a computer-implemented method for detecting heart rate signals. The method includes a step of capturing a first image and a second image of a region of interest. The second image is aligned with the first image in a first spectrum that is insensitive to a characteristic to obtain a correction. Then, a correction based upon the above-mentioned alignment is applied to the second image in a second spectrum that is sensitive to the characteristic. Additional aspects described an image capturing device and a computer-implemented method for updating a user-interactive activity.

### BACKGROUND

One way to assess health and fitness is to measure a person's heart rate frequency, i.e., pulse rate. The measured pulse rate may reveal certain susceptibilities to heart-related concerns including, for example, arthrosclerosis, hypertension and diabetes. The ability to manage heart rate frequency within predefined limits according to criteria such as age, sex and weight can help promote healthy, happier and longer lives.

Over the years, various non-invasive imaging techniques have been developed to analyze the heart. For example, computed tomography employs x-rays to view the heart. Another technique includes magnetic resonance imaging (MRI). A further cardiac imaging technique uses ultrasound. While each of these techniques is non-invasive, they require little or no movement of the patient. Otherwise, the accuracy of the reading may be compromised.

More recently, non-invasive and non-contact imaging techniques have been utilized for cardiac imaging. In particular, cameras with an optical lens may capture images of a region-of-interest (ROI). Local image matching employing optical flow techniques is then performed to align multiple, successive captured images. However, these techniques are susceptible to undesirable noise which ultimately degrades the signal to noise ratio.

### SUMMARY

This summary is provided to introduce a selection of concepts in a simplified form that is further described below in the Detailed Description. This Summary is not intended to limit the scope of the claimed subject matter. The foregoing needs are met, to a great extent, by the present application directed to a process and apparatus for heart rate detection.

A computer-implemented method is disclosed in an aspect of the application. The method includes a step of capturing a first image and a second image of a region of interest. The second image is aligned with the first image in a first spectrum that is sensitive to a characteristic to obtain a correction. Then, a correction based upon the above-mentioned alignment is applied to the second image in a second spectrum that is sensitive to the characteristic.

Another aspect describes an image capture device. The device includes an image capture component that captures at least two successive image pairs of a region of interest. The devices also includes a memory that stores the at least two successive image pairs captured by the image capture component. The device also includes a processor, operably coupled to the memory and the image capture component. The processor is configured to perform the instructions of identifying a first spectrum that is not sensitive to a characteristic and a second spectrum that is sensitive to the characteristic for the at least two successive image pairs. The processor is also configured to perform the instructions of measuring intensity of a heart rate based upon a computer-generated signal based on data received in the second spectrum for the at least two successive image pairs.

A computer-implemented system is described in even another aspect of the application. The system may include a means for capturing images. In one aspect, this may be an image capture device as described above. The system may also include a computing device operably connected to the image capture device.

A computer-implemented method for updating a user-interactive activity is described in yet even another aspect of the application. The method includes receiving at least two successive image pairs for a region of interest. The method also includes determining a heart rate signal based upon the received image pairs. The method further includes the step of updating the user-interactive activity based upon the determined heart rate signal.

There has thus been outlined, rather broadly, certain aspects in order that the detailed description thereof may be better understood, and in order that the present contribution to the art may be better appreciated. There are, of course, additional aspects that will be described below and which will form the subject matter of the claims appended hereto.

### BRIEF DESCRIPTION OF THE DRAWINGS

In order to facilitate a more robust understanding of the application, reference is now made to the accompanying drawings, in which like elements are referenced with like numerals. These drawings should not be construed to limit the application and are intended only to be illustrative.

FIG. 1 illustrates an example computing environment for detecting a user's heart rate according to an aspect.

FIG. 2 illustrates an exemplary capture device and computing system used to detect a user's heart rate according to an aspect.

FIG. 3 illustrates an exemplary multimedia console used in combination with the capture device according to an aspect.

FIG. 4 illustrates an exemplary capture device in communication with a network and server according to another aspect.

FIG. 5 illustrates an exemplary display of detected heart rate for a user according to an aspect.

FIG. 6 illustrates a flowchart of an exemplary illustrative method for detecting computer-generated signals received from an image according to an aspect.

FIG. 7 illustrates a flowchart of an exemplary illustrative method of updating an interactive activity based upon heart rate detection according to an aspect.

### DETAILED DESCRIPTION

A detailed description will be discussed in reference to various figures, aspects and aspects herein. Although this description provides detailed examples of possible implementations, it should be noted that the details are intended to be examples and thus do not limit the scope of the technology described in this application.

Reference in this specification to “one aspect,” “an aspect,” “one or more aspects,” “an aspect” or the like means that a particular feature, structure, or characteristic described in connection with the aspect is included in at least one aspect of the application. Moreover, the term “aspect” in various places in the specification is not necessarily referring to the same aspect. That is, various features are described which may be exhibited by some aspects and not by others.

The apparatuses, systems and methods described in this application more accurately detect computer-generated signals captured from a region of interest (ROI) including but not limited to a human. In one aspect, a more accurate detection of heart rate signals is disclosed. In particular, measurements may continuously be taken in real-time. Moreover, measurements may be taken in a contactless environment. That is, a user or patient is not physically connected to a detection device. Separately, the user is not required to limit range of motion while measurements are being taken. Accordingly, a user may engage in physical activity within a ROI while obtaining an accurate detection of his heart rate.

According to one aspect, an apparatus is disclosed for measuring computer-generated signals captured from one or more images of a particular ROI. In one aspect, the one or more images are captured by a camera. Computer-generated signals are detected from the one or more images and used to perform alignment, e.g., registration. By so doing, per-frame motions including but not limited to talking and blinking can be corrected. Thereafter, the corrected signal can be applied to a second signal from the second or successive captured image. An intensity of the second signal is measured. Preferably, the measured intensity provides an estimated heart rate, i.e., pulse rate.

According to one example, a series of images are taken, e.g., a 5 second window of time at 30 hertz, yielding 147 images. 147 individual values were obtained. Pairs of the images are obtained, e.g., the first and fourth images, then the second and fifth images, etc., and 147 alignments are performed. Then, subtraction per pixel of the value in the spectrum being observed, e.g., green, is performed to yield a delta value per pixel. This is performed for all pixels in the region of interest (ROI). The values are averaged to generate an overall value for this image pair. In an aspect, a per-pixel weight may be applied which affects how much each pixel affects the final computed average. Next, a standard signal analysis is performed for all image pairs. By so doing, the heartrate value for this window of time, e.g., 5 seconds is obtained. Thus, an average color change value over a succession of image pairs is employed to derive the heart rate signal.

According to another aspect of the application, interactive techniques and systems are disclosed which allow the user to improve their strength, endurance and overall fitness in real-time. In one aspect, for example, the user's heart rate is continuously monitored by the system. The system recognizes the target heart rate or predetermined fitness goals of the user and updates the physical activity requested by the user in real-time. This may include, for example, changing a fitness module provided by the console. By so doing, the user may increase or decrease his heart rate to align with predefined fitness goals. For example, if the user wishes to build endurance, they may opt for a lower target heart rate zone based upon a maximum heart rate. On the other hand, if the user wishes to lose weight, they may opt for a higher target heart rate zone based upon a maximum heart rate. The system determines the user's current heart rate and compares it with the predefined user target heart rate input. The system updates the fitness module to maintain, increase or decrease the workout to align the current heart rate detected with the ideal heart rate.

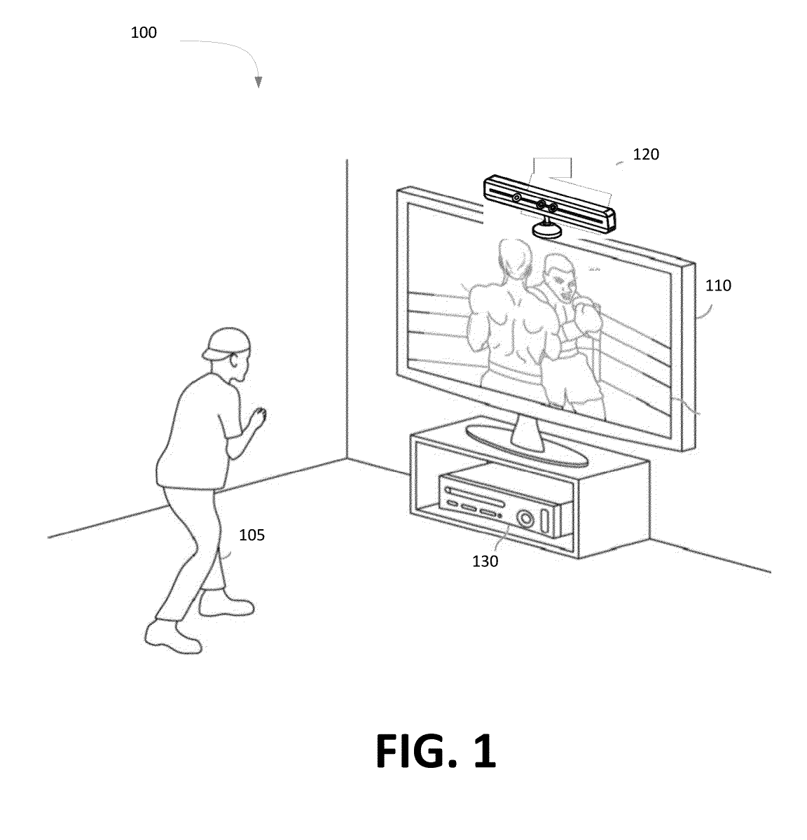

FIG. 1 illustrates an environment 100 in which aspects of the apparatus and methods disclosed in this application may be utilized. The environment 100 may include but is not limited to a room in a home. A user 150 is shown as being located inside the environment 10. A capture device 120 and a computing device 130 may also be located in the environment 100. In one aspect, the capture device 120 and computing device 130 are housed in a single, unitary device. Alternatively, the capture device 120 and computing device 130 may be separate and independent apparatuses.

The capture device 120 may be used to obtain depth images and color images, also known as RGB images. In an example, two cameras such as a RGB camera and an IR camera may be employed. The spectrum is the portion of the light spectrum captured in the image. In one example, a green portion of the spectrum may be filtered and used to align the image.

The capture device 120 may be, for example, a camera that may be used to visually monitor one or more users, such as user 105. The capture device 120 may also include a laptop, personal digital assistant (PDA), tablet, or cellular telephone to provide data that is readily processed and converted into an accurate determination of the heart rate for a given individual.

It will be appreciated that these results are readily achieved in many cases with only a relatively few consecutive images. For example, as few as 100 to 200 such images, captured, for example, at 10 to 30 frames per second over about 5 to 10 seconds, may be sufficient to provide a relatively accurate determination of one's heartbeat.

In one aspect, the capture device 120 can receive a plurality of visible light images corresponding to a subject's skin proximal to a blood-transporting capillary and then process that plurality of visible light images to thereby determine a heart rate for the subject. As used herein, reference to “visible light images” will be understood to refer to photographs, including, perhaps most typically, digital photographs, of the kind that are normally associated with an ordinary visible light image capture component such as a camera. This can include, as noted below, normal color images as well as essentially any other visible light photographic format including, but not limited to, the RGB format, the hue saturation value (HSV) format, a grayscale format, and so forth. In one example, these visible light images will have a size/resolution of at least 100×100 pixels with 176×144-sized images serving very well in this regard and with higher resolution and/or sized pictures.

The visible light images can comprise images corresponding to color components. In such a case, the aforementioned processing may comprise transforming the corresponding color components into different color components. For example, this can comprise providing RGB-based images and transforming those images into images comprised of HSV components. The dimensional content of these high contrast images can then be reduced to provide corresponding reduced-dimensionality images. The latter can comprise, for example, selecting and using only one image component from amongst a plurality of image components that comprise the corresponding high contrast images.

Further, the environment may include a display 110. The display allows for the signals captured by the capture device 120 and processed by the computing device 130 to be viewed by the user 105. The display 110 may include but is not limited to a computer monitor or television set. The display is also able to recognize the user 105 for which optical measurements are being taken of Frames may then be displayed by the display device 110. The display device 110 may or may not be partially transparent. For example, the display device 110 may be configured to display both the image captured by the capture device 120 along with augmentations

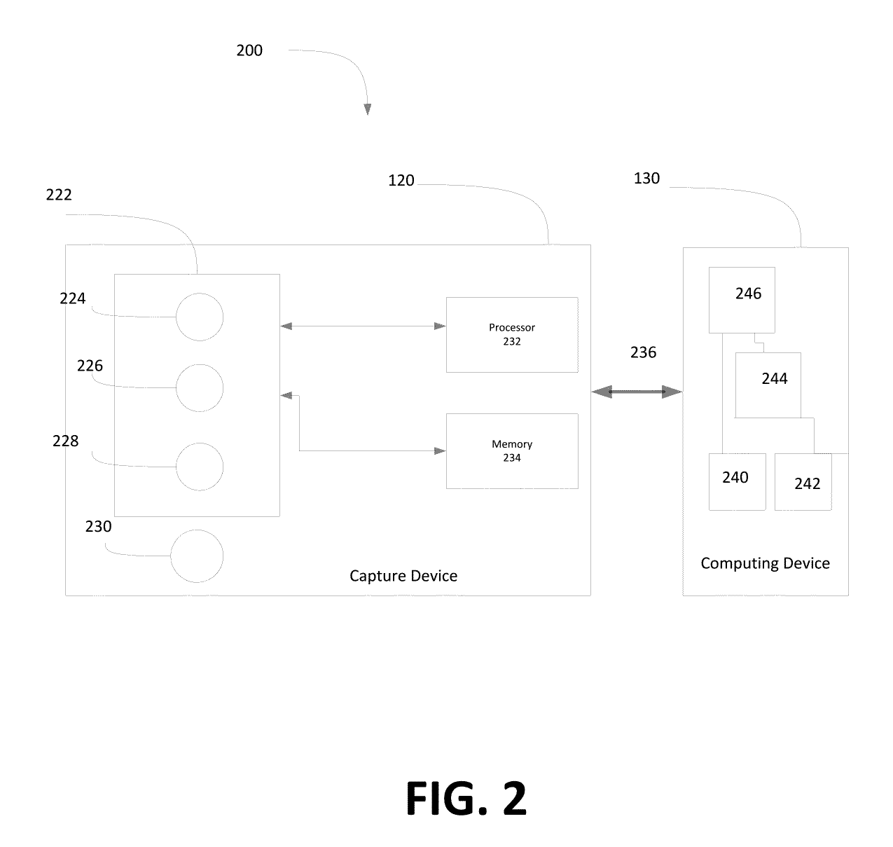

As shown in FIG. 2, the capture device 120 may include an image camera component 222. In one aspect, the image camera component 222 may be a depth camera that may capture a depth image of a scene. The depth image may include a two-dimensional (2-D) pixel area of the captured scene where each pixel in the 2-D pixel area may represent a depth value such as a distance in, for example, centimeters, millimeters, or the like of an object in the captured scene from the camera.

As shown in FIG. 2, the image camera component 222 may include an infra-red (IR) light component 224, a three-dimensional (3-D) camera 226, and an RGB camera 228 that may be used to capture the depth image of a scene. For example, in time-of-flight analysis, the IR light component 224 of the capture device 120 may emit an infrared light onto the scene and may then use sensors (not shown) to detect the backscattered light from the surface of one or more targets and objects in the scene using, for example, the 3-D camera 226 and/or the RGB camera 228.

In some aspects, pulsed infrared light may be used such that the time between an outgoing light pulse and a corresponding incoming light pulse may be measured and used to determine a physical distance from the capture device 120 to a particular location on the targets or objects in the scene. Additionally, in other example aspects, the phase of the outgoing light wave may be compared to the phase of the incoming light wave to determine a phase shift. The phase shift may then be used to determine a physical distance from the capture device to a particular location on the targets or objects.

According to another example aspect, time-of-flight analysis may be used to indirectly determine a physical distance from the capture device 120 to a particular location on the targets or objects by analyzing the intensity of the reflected beam of light over time via various techniques including, for example, shuttered light pulse imaging.

In another example aspect, the capture device 120 may use a structured light to capture depth information. In such an analysis, patterned light, i.e., light displayed as a known pattern such as grid pattern, a stripe pattern, or different pattern, may be projected onto the scene via, for example, the IR light component 224. Upon striking the surface of one or more targets or objects in the scene, the pattern may become deformed in response. Such a deformation of the pattern may be captured by, for example, the 3-D camera 226 and/or the RGB camera 28 and may then be analyzed to determine a physical distance from the capture device to a particular location on the targets or objects. In some implementations, the IR Light component 224 is displaced from the cameras 226 and 228 so triangulation can be used to determined distance from cameras 226 and 228. In some implementations, the capture device 120 will include a dedicated IR sensor to sense the IR light.

The capture device 120 may further include a processor 232 that may be in operative communication with the image camera component 222. The processor 232 may include a standardized processor, a specialized processor, a microprocessor, or the like that may execute instructions including, for example, instructions for receiving a depth image, generating the appropriate data format, e.g., frame, and transmitting the data to computing device 112.

The capture device 120 may further include a memory component 234 that may store the instructions that may be executed by the processor 232, images or frames of images captured by the 3-D camera and/or RGB camera, or any other suitable information, images, or the like. According to an example aspect, the memory component 234 may include random access memory (RAM), read only memory (ROM), cache, Flash memory, a hard disk, or any other suitable storage component. In one aspect, the memory component 234 may be a separate component in communication with the image capture component 222 and the processor 232. According to another aspect, the memory component 234 may be integrated into the processor 232 and/or the image capture component 222.

As also shown in FIG. 2, the capture device 120 may be in communication with the computing device 130 via a communication link 236. The communication link 236 may be a wired connection including, for example, a USB connection, a Firewire connection, an Ethernet cable connection, or the like, and/or a wireless connection such as a wireless 802.11b, g, a, or n connection.

In one aspect, the computing device 130 may provide a timer to the capture device 120 that may be used to determine when to capture, for example, a facial representation via the communication link 236. Additionally, the capture device 120 provides the depth images and color images captured by, for example, the 3-D camera 226 and/or the RGB camera 228 to the computing device 112 via the communication link 236. In one aspect, the depth images and color images are transmitted at 30 frames per second. The computing device 130 may then use the model, depth information, and captured images to, for example, control an application such as a game or word processor and/or animate an avatar or on-screen character.

Also illustrated in FIG. 2 is a detailed view of the computing device 130. The computing device 130 may include a gestures library 240, structure data 242, depth image processing and object reporting module 244 and application 246. Depth image processing and object reporting module 244 use the depth images to track motion of objects, such as the user and other objects. To assist in the tracking of the objects, depth image processing and object reporting module 244 use the gestures library 240 and structure data 242.

Structure data 242 includes structural information about objects that may be tracked. For example, a skeletal model of a human may be stored to help understand movements of the user and recognize body parts. Structural information about inanimate objects may also be stored to help recognize those objects and help understand movement.

Gestures library 240 may include a collection of gesture filters, each comprising information concerning a gesture that may be performed by the skeletal model (as the user moves). The data captured by the cameras 226, 228 and the capture device 120 in the form of the skeletal model and movements associated with it may be compared to the gesture filters in the gesture library 240 to identify when a user (as represented by the skeletal model) has performed one or more gestures. Those gestures may be associated with various controls of an application. Thus, the computing system 130 may use the gestures library 240 to interpret movements of the skeletal model and to control application 246 based on the movements. As such, gestures library may be used by depth image processing and object reporting module 244 and application 246.

Application 246 may also be a video game, fitness module, productivity application, etc. In one aspect, depth image processing and object reporting module 244 will report to application 246 of identification of each object detected and the location of the object for each frame. Application 246 will use that information to update the position or movement of an avatar or other images in the display.

According to another aspect, the computing device 130 may also be configured to run or include an entity, e.g., software, causing the hardware of the computing device 130 to perform operations. The computing device 130 may include a computer-readable medium that may be configured to maintain instructions that cause the computing device 130, and more particularly hardware of the computing device 130 to perform operations. Thus, the instructions function to configure the hardware to perform the operations and in this way result in transformation of the hardware to perform functions. The instructions may be provided by the computer-readable medium to the computing device 130 through a variety of different configurations.

In an exemplary aspect, the computing device 130 may be a computer, a gaming system or console, or the like. The images may be used by the computing device 130 to identify one or more users or ROIs, as well as to track motion and/or other user behaviors. In one aspect, the tracked motion and/or other signals, included heart rate, may be used to update an application. As discussed above, the tracked motion and/or other signal may be determined and compared with predefined user acceptability limits. For example, the acceptability limits may include a certain heart rate target zone. The computing device 130 may alter the game, fitness module or the like to ensure the user's heart rate is maintained within the target heart rate zone for a predetermined period of time.

According to another aspect, the computing device 130 may also include an integrated circuit. The integrated circuit may include an application specific integrated circuit (ASIC) used for natural user interface processing. The integrated circuit may be part of a natural user interface system which may include a plurality of sensors and/or illuminators. The integrated circuit may be embedded in a host processor and/or data may be transferred from the integrated circuit to the host processor to perform some of the user interface processing. Additionally data may be transferred from the host processor to the integrated circuit to perform some of the user interface processing or to add more information to be processed in the integrated circuit. The natural user interface features may include, for example, any action made by a user's body, through the user's body parts or by objects held, movable and/or controlled by a user, which may be detected and processed by the system according to aspects of the application to produce a user interface event.

For example, natural user interface feature may include a finger movement, body gesture, head movement, gaze, face changes, movement and/or change in expression, voicing, speaking, object movement and/or any other suitable natural user interface feature. Data processing dedicated areas may obtain the plurality of user interface features based on scene features detected by a plurality of sensors, for example within a defined period of time, for example, a predefined or pre-known period of time such as, for example, the frame period of the imaging device or another defined period of time. The scene features may include, for example, any sensual feature that may be detected in/from a scene. For example, the scene features may include any visual feature, auditory feature, and/or features pertaining to smell, taste and/or pressure that may be detected in/from a scene. For example, the scene features may include depth, image, color, illumination, sound, and/or any other suitable feature.

In one aspect, the computer-readable medium is a signal bearing medium. That is, it is configured to transmit instructions, e.g., as a carrier wave, to the hardware of the computing device, such as via the network 104. The computer-readable medium may also be configured as a computer-readable storage medium and is therefore an article of manufacture. That is, the computer-readable storage medium includes a concrete, tangible physical structure. Any computer-readable storage medium described in the application is not to be construed as a signal such as transient or propagating signals.

Examples of a computer-readable storage medium include a random-access memory (RAM), read-only memory (ROM), an optical disc, flash memory, hard disk memory, and other memory devices that may use magnetic, optical, and other techniques to store instructions and other data.

According to another aspect of the application, a computer-implemented method is disclosed for detecting a signal from a captured image. The images are illustrated as being separated into a plurality of different color channels, examples of which include a red color channel 202, a green color channel and a blue color channel.

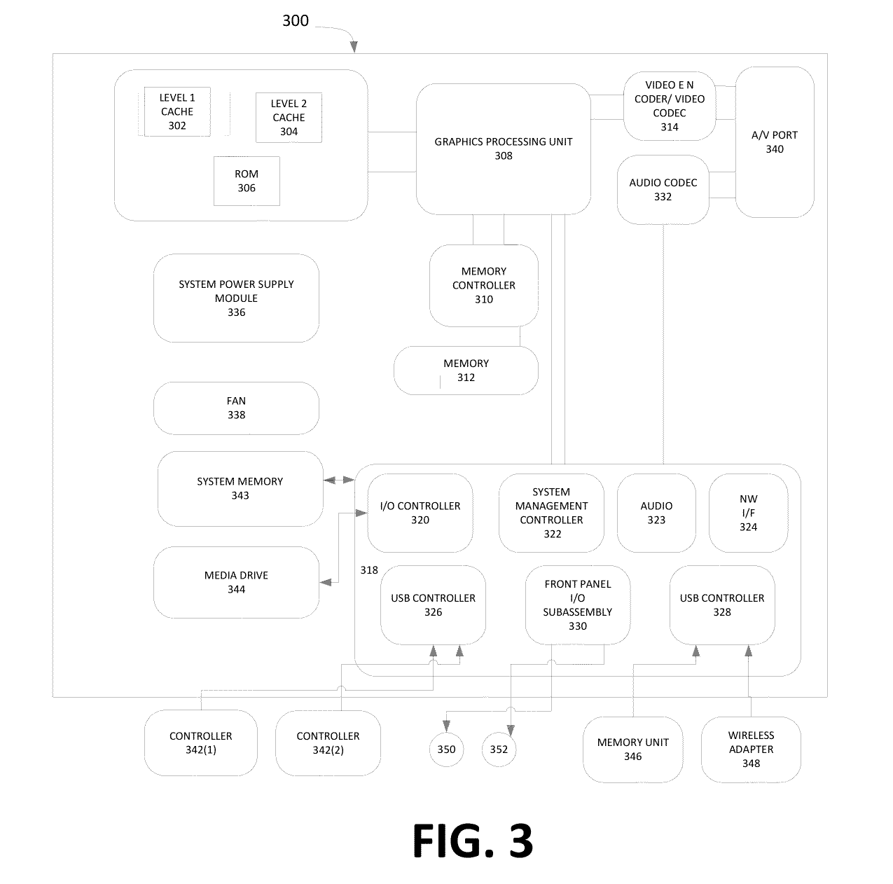

FIG. 3 illustrates an aspect of a computing system that may be the computing system 130 as shown in FIGS. 1 and 2 used to track motion and/or animate (or otherwise update) an avatar or other on-screen object displayed by an application. The computing system such as the computing system 130 described above may be a multimedia console, such as a gaming console. As shown in FIG. 3, the multimedia console 300 has a central processing unit (CPU) 301 having a level 1 cache 102, a level 2 cache 304, and a flash ROM (Read Only Memory) 306. The level 1 cache 302 and a level 2 cache 304 temporarily store data and hence reduce the number of memory access cycles, thereby improving processing speed and throughput. The CPU 301 may be provided having more than one core, and thus, additional level 1 and level 2 caches 302 and 304. The flash ROM 306 may store executable code that is loaded during an initial phase of a boot process when the multimedia console 300 is powered ON.

A graphics processing unit (GPU) 308 and a video encoder/video codec (coder/decoder) 314 form a video processing pipeline for high speed and high resolution graphics processing. Data is carried from the graphics processing unit 308 to the video encoder/video codec 314 via a bus. The video processing pipeline outputs data to an A/V (audio/video) port 340 for transmission to a television or other display. A memory controller 310 is connected to the GPU 308 to facilitate processor access to various types of memory 312, such as, but not limited to, a RAM (Random Access Memory).

The multimedia console 300 includes an I/O controller 320, a system management controller 322, an audio processing unit 323, a network interface 324, a first USB host controller 326, a second USB controller 328 and a front panel I/O subassembly 330 that are preferably implemented on a module 318. The USB controllers 326 and 328 serve as hosts for peripheral controllers 342(1)-342(2), a wireless adapter 348, and an external memory device 346 (e.g., flash memory, external CD/DVD ROM drive, removable media, etc.). The network interface 324 and/or wireless adapter 348 provide access to a network, e.g., the Internet, home network, etc., and may be any of a wide variety of various wired or wireless adapter components including an Ethernet card, a modem, a Bluetooth module, a cable modem, and the like.

System memory 343 is provided to store application data that is loaded during the boot process. A media drive 344 is provided and may comprise a DVD/CD drive, Blu-Ray drive, hard disk drive, or other removable media drive, etc. The media drive 344 may be internal or external to the multimedia console 300. Application data may be accessed via the media drive 344 for execution, playback, etc. by the multimedia console 300. The media drive 344 is connected to the I/O controller 320 via a bus, such as a Serial ATA bus or other high speed connection (e.g., IEEE 1394).

The system management controller 322 provides a variety of service functions related to assuring availability of the multimedia console 300. The audio processing unit 323 and an audio codec 332 form a corresponding audio processing pipeline with high fidelity and stereo processing. Audio data is carried between the audio processing unit 323 and the audio codec 332 via a communication link. The audio processing pipeline outputs data to the A/V port 340 for reproduction by an external audio player or device having audio capabilities.

The front panel I/O subassembly 330 supports the functionality of the power button 350 and the eject button 352, as well as any LEDs (light emitting diodes) or other indicators exposed on the outer surface of the multimedia console 300. A system power supply module 336 provides power to the components of the multimedia console 300. A fan 338 cools the circuitry within the multimedia console 300.

The CPU 301, GPU 308, memory controller 310, and various other components within the multimedia console 300 are interconnected via one or more buses, including serial and parallel buses, a memory bus, a peripheral bus, and a processor or local bus using any of a variety of bus architectures. By way of example, such architectures can include a Peripheral Component Interconnects (PCI) bus, PCI-Express bus, etc.

When the multimedia console 300 is powered ON, application data may be loaded from the system memory 343 into memory 312 and/or caches 302, 304 and executed on the CPU 301. The application may present a graphical user interface that provides a consistent user experience when navigating to different media types available on the multimedia console 300. In operation, applications and/or other media contained within the media drive 344 may be launched or played from the media drive 344 to provide additional functionalities to the multimedia console 300.

The multimedia console 300 may be operated as a standalone system by simply connecting the system to a television or other display. In this standalone mode, the multimedia console 300 allows one or more users to interact with the system, watch movies, or listen to music. However, with the integration of broadband connectivity made available through the network interface 324 or the wireless adapter 348, the multimedia console 300 may further be operated as a participant in a larger network community.

When the multimedia console 300 is powered ON, a set amount of hardware resources are reserved for system use by the multimedia console operating system. These resources may include a reservation of memory, e.g., 16 MB, CPU and GPU cycles. e.g., 5%, networking bandwidth, e.g., 8 Kbps, etc. Because these resources are reserved at system boot time, the reserved resources do not exist from the application's view.

With regard to the GPU reservation, lightweight messages generated by the system applications, e.g., popups, are displayed by using a GPU interrupt to schedule code to render popup into an overlay. The amount of memory required for an overlay depends on the overlay area size and the overlay preferably scales with screen resolution. Where a full user interface is used by the concurrent system application, it is preferable to use a resolution independent of application resolution. A scaler may be used to set this resolution such that the need to change frequency and cause a TV resynch is eliminated.

After the multimedia console 300 boots and system resources are reserved, concurrent system applications execute to provide system functionalities. The system functionalities are encapsulated in a set of system applications that execute within the reserved system resources described above. The operating system kernel identifies threads that are system application threads versus gaming application threads. The system applications are preferably scheduled to run on the CPU 301 at predetermined times and intervals in order to provide a consistent system resource view to the application. The scheduling is to minimize cache disruption for the gaming application running on the console.

When a concurrent system application requires audio, audio processing is scheduled asynchronously to the gaming application due to time sensitivity. A multimedia console application manager (described below) controls the gaming application audio level (e.g., mute, attenuate) when system applications are active.

Input devices (e.g., controllers 342(1) and 342(2)) are shared by gaming applications and system applications. The input devices are not reserved resources, but are to be switched between system applications and the gaming application such that each will have a focus of the device. The application manager preferably controls the switching of input stream, without knowledge the gaming application's knowledge and a driver maintains state information regarding focus switches. The cameras 226, 228 and capture device 120 may define additional input devices for the console 300 via USB controller 326 or other interface.

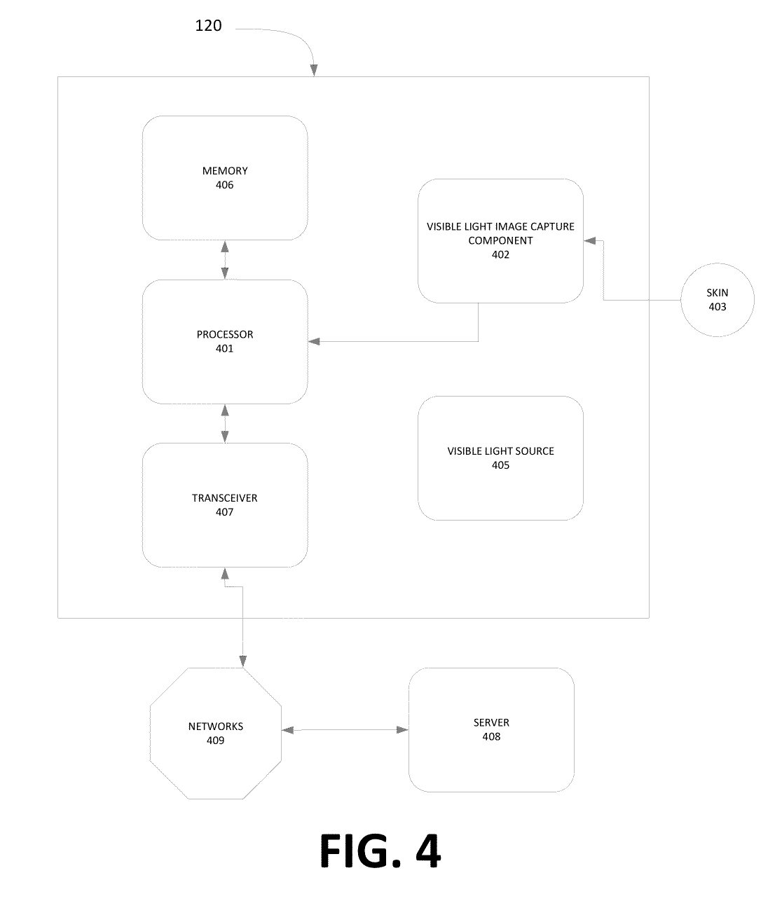

According to a further aspect as shown in FIG. 4, the capture device 120 is able to capture, track and analyze movement and/or gestures of the user. Accordingly, these can be used as controls to update an application or game running on a game console 300. In this aspect, the capture device 120 may further comprise a memory 406 that is operably coupled to the processor 401. This memory 406 can serve to store, temporarily or permanently, the visible light images, the interim image processing results, and/or the determined heart beat information. This memory 406 can also serve to store the operating instructions that permit the processor 401 to carry out the described steps.

The capture device 120 also includes a visible light image capture component 402. The capture component 402 preferably captures light from a user's skin 403. The capture device 120 may also include a visible light source 405. The visible light source 405 helps illuminate the subject or ROI in order for the capture component 402 to take images that ultimately are processed by the processor 401.

Moreover, the capture device 120 may have two-way wireless communication capability. To facilitate such a capability, the apparatus 120 may include a transceiver 407 to communicate with a server 408, e.g., the Cloud, via one or more intervening networks 409. The intervening networks may include but are limited to a local area network, cellular telephone network and the Internet. The visible light images captured by the capture component 402 may also be sent to the server 408. The images may either be in their original or partially processed form.

The server 408 may either be located locally or remote to the capture device 120. The server 408 can perform comparisons between the detected heart rate and a predetermined user input. The server 408 then returns information to the capture device 120, upon reviewing the game or fitness module being played by the user. If the measured heart rate is outside of the predetermined heart rate zone, the server 408 can connect to the game or fitness module being played by the user and perform an update as necessary. The update causes the display to change the activity shown on a display to the user. The update may also cause the sound to change in accordance with the updates. By so doing, the user is able to receive an effective and efficient workout matching with their predetermined target heart rate. For example, if the user was playing a boxing game or involved in a cardio kickboxing workout, his or her heart rate can be monitored along with other criteria, including number of punches thrown/lands and kicks. By so doing, the user's performance, skill and fitness level can all be measured and provided to the user for monitoring.

A non-transitory computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

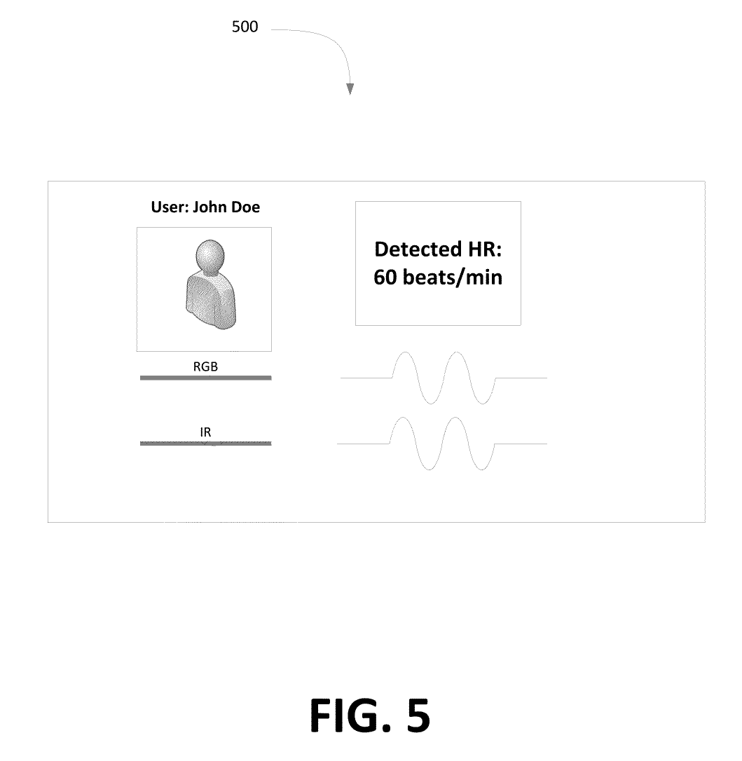

According to another aspect as shown in FIG. 5, information related to a user's performance and fitness level are graphed, saved and provided to a user in a display 500. The display can be provided on any user-interface including but not limited to a television, laptop, tablet, cell phone, or wearable display technology, such as eyeglasses e.g., Google glass or a watch, e.g., Apple Watch.

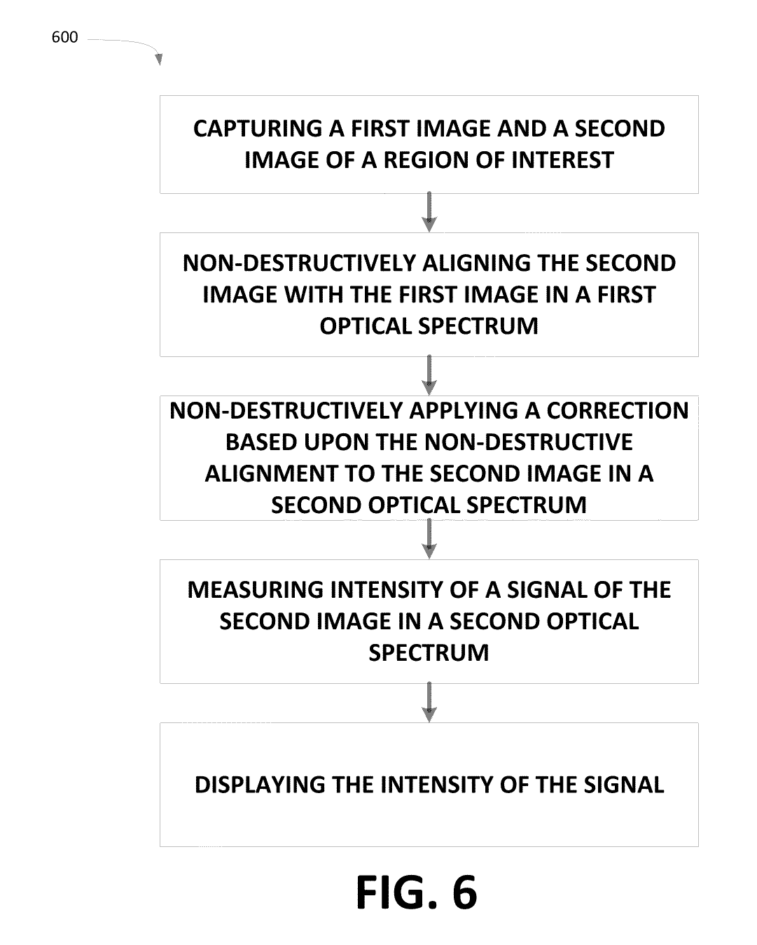

According to another aspect of the application, a computer-implemented method for measuring an intensity of a computer-generated signal is disclosed. The method is provided in the flowchart 600 illustrated in FIG. 6.

In one aspect, a first and a second image are received. The second image is preferably a successive image of the first image. Preferably, the images are of a human user. More preferably, the images are of skin. Even more preferably, the images are of the skin on a face of a human. The first and second images are generally in different spatial orientations from each other. That is, while both images may be of the face, the second image may be slightly different caused by movement, physical exercise, etc. As discussed above, micro-fluctuations in color of the face are captured by a capture device 130, such as a camera, and each image is evaluated in more detail.

The images are generally received within a predefined area such as the environment 100 shown in FIG. 1. In one aspect, the images are taken from less than about 10 feet away. In another aspect, the images are taken at least about 2 feet away from the camera. In yet another aspect, the images are taken about 2 to 10 feet away from the camera.

Next, the captured images are separated into different spectrums. For example, the image may include spectrums such as different color channels, e.g., red, green or blue, infrared, or ultraviolet.

The first image is aligned with the second image within a first spectrum. For example, the image may be broken up into separate color channels such as a red, blue and green color channel. Each of the color channels may represent a spectrum. In one aspect, the alignment may be performed in the red color channel. In another aspect, the alignment may be performed in the blue color channel. As an example, it has been determined that the red and blue color channels emit a very weak heart rate signal. As another example, the green color channel has been determined to emit a strong heart rate signal. Since the skin is so richly perfused, the pulsatile component of the cardiac cycle may be detected via recording changes in intensity of the skin color over time. The direct current (DC) bias of the signal component relates to the underlying intensity of the skin in the characteristic spectrum and can be attributable to bulk absorption of the skin tissue. Meanwhile, the alternating current (AC) bias of the signal relates to variations in blood volume in the skin caused by the pressure pulse of the cardiac cycle. The height of the AC component of the signal is proportional to the pulse pressure. This is the difference between the systolic and diastolic pressure in the arteries.

Non-destructively aligning is understood to mean that the raw data is not altered with respect to color intensity even though the position of the pixels may be altered. Moreover, data that may be destroyed during image alignment does not include heartrate information. During the alignment step, e.g., registration, a correction for movement is determined between the first and the second image in a first spectrum. Again, the spectrum in which alignment is performed has a weak intensity signal for heart rate.

Thereafter, the correction obtained from the aligning step is applied to the second image in a second spectrum. For example, the red or blue channel may be used to align the current image with a previous, cached image as perfectly as possible. In one aspect, a per-pixel comparison of the decomposed, aligned, skin-only image of the current frame's head is performed against a cached, decomposed version of the previous frame's head. Those pixels having deltas between the previous frame's head and current frame's head that are determined to be trending in a consistent direction, and are within a given number of standard deviations of brightness, are summed and averaged. A measure of noisiness is generated based on how far the pixel is from the average delta and brightness.

In one example, on a per-pixel basis, the correction is applied to the current and previous image in the green channel. Optical flow techniques may be used to determine how one image can be warped to align with the other. The results of the optical flow yields a set of pixel deltas to move one image to align with the other. For example, if the pixel of the current image in the green channel is equal to x=35, y=27 and needs to move left 3 pixels to match the previous image in the red color channel, then the current image in the green color channel at pixel x=35, y=27 would have a “left 3 pixels” value. Thus, alignment can be performed by generating a correction in a spectrum that does not easily detect data representative of a characteristic, such as for example, blood flowing around the face, which is then applied to the captured image, either the first or the second, in a different spectrum that is sensitive to data representative of the characteristic, e.g., blood flowing around the face. By so doing, an accurate heart rate signal is obtained without inadvertently correcting for signals attributed to the characteristic, e.g., blood flowing around the face. It is envisaged within this application that the data/characteristic can be blood flowing around any extremity of the body.

In one example, the received signal from the successive image pairs may be transformed into the frequency space via a Fourier transform, Lomb-Scargle Spectral-Analysis Periodigram or the like for analysis. The frequency obtained from the signal of the successive image pairs may be graphed and ultimately displayed to the user as shown in FIG. 5.

In an exemplary aspect, the second spectrum is a second color channel different from the first color channel. Even further, the second color channel is the green color channel.

Further the signal intensity may be transmitted to a display. By so doing, the user may be able to monitor certain aspects, such as for example, heart rate. The display may include but is not limited to a television, laptop, desktop monitor, tablet, cellphone, wearable technology having a display.

In another aspect, the detection of heart rate may be employed to update user-interactive activity in relation to a gaming system console. The gaming console may include games—single and multiplayer—for interacting through the internet, for television viewing, fitness videos, etc. In an aspect, the fitness videos may be accessible via the Internet. Moreover, the videos may be interactive. That is, the workouts are optimized to provide instant feedback on form, power and heart rate using precision technology. The technology may include pulse detection, muscle mapping and an energy meter. For example, the fitness modules may provide workout modules, recommendations, challenges, performance history, rewards and achievements to keep the user motivated.

In an aspect, the system includes a sensor for reading the distribution of weight and the explosiveness of movements. The sensor also is able to detect and provide an analysis with the help of its processor to analyze the area of the body being worked. In one aspect, the pulse may be detected via micro-fluctuations in the skin, preferably in the face, to estimate heart rate. Moreover, the sensor is also able to map muscles to identify which muscles are most engaged by the power, force and transfer of weight in the body. The processor interacts with the fitness video and is a able to provide tips on how to produce stronger results. In another aspect, the system tracks the quality of the user's performance by measuring balance, tempo and form.

In another aspect, the system is configured to provide smart recommendations. The smart recommendations account for workout histories and past performance. The smart recommendations may also account for selected workouts by all fitness users who may be connected to a previous service provider, such as for example, Xbox Fitness. The smart recommendations feature also offers a variety of workouts of different lengths. By so doing, it is easy for a user to jump into a workout based upon available time.

The system also is configured to provide challenges to the user. The challenges are geared to push the user to their physical limits, work harder and stay motivated.

Moreover, the technology also provides personalized and social challenges. Discuss interaction of heart rate with gaming console with a XBOX live.

According to a further aspect of the application, a computer-implemented method for updating a user's activity is described. More particularly, interactive techniques and systems are disclosed which allow the user to improve their strength, endurance and overall fitness in real-time. For example, an interactive activity is displayed to the user on a display. The display may include but is not limited to a television, smartphone, laptop, tablet or personal digital assistant.

The system receives feedback from a user who is performing an interactive activity provided by a gaming console. The feedback may include, for example, heart rate signals. The feedback may be provided in a contactless environment. Preferably the detection of heart rate is provided less than 10 feet from a sensor. The detection may also be provided between 2 and 10 feet from the sensor. As discussed above in relation to FIG. 4, the feedback includes capturing images of a ROI and performing an alignment with at least two images in a first spectrum, and then applying the correction obtained in the first spectrum to a second image in second color channel.

After receiving a real-time detection of heart rate of a user, the system may update the interactive activity being performed by the user based upon predetermined conditions. As discussed above, the interactive activity may include fitness modules. By so doing, the user's strength, endurance and overall fitness may be enhanced. Updating the fitness modules may be afford the user with a personal trainer without having to spend money and time going to a gym for one-on-one supervision. Instead, the user may be able to work out in the convenience of his or her own home. Moreover, the user is able to obtain accurate detection of his heart rate which is essential for improving physical fitness results.

Predetermined conditions include a target heart rate zone supplied by the user in advance of beginning the interactive activity. By so doing, the user is able to maintain his heart rate within a specific zone. Moreover, the user may be able to increase the level of activity based upon predetermine conditions. The predetermined condition may include for example, the user's input to increase or decrease the intensity level based upon the user's ultimate fitness goals.

In one aspect, for example, the user's heart rate is continuously monitored by the system. Alternatively, the system periodically measures the user's heart rate. Thus, the system may be configured to recognizes the target heart rate or predetermined fitness goals of the user and updates the physical activity requested by the user in real-time or at specific intervals. The specific intervals may include after a particular workout routine is completed. By so doing, the user can complete a set of exercises without having to stop in the middle of the workout or set and begin an entirely new fitness module.

According to an aspect, the console the user may increase or decrease his heart rate to align with predefined fitness goals. For example, if the user wishes to build endurance, they may opt for a lower target heart rate zone based upon a maximum heart rate. On the other hand, if the user wishes to lose weight, they may opt for a higher target heart rate zone based upon a maximum heart rate. The system determines the user's current heart rate and compares it with the predefined user target heart rate input. The system updates the fitness module to maintain, increase or decrease the workout to align the current heart rate detected with the ideal heart rate.

According to yet another aspect of the application, non-transitory computer-readable or executable storage medium for storing computer-readable or executable instructions is disclosed. The medium may be volatile or non-volatiles, removable and non-removable media implemented in any method or technology for storage of information such as a computer-readable instructions, data structures, program modules, or other data. For example, computer-readable media includes, but is not limited to, RAM, ROM, EPROM (erasable programmable read only memory), EEPROM (electrically erasable programmable read only memory), Flash memory or other solid state memory technology, CD-ROM, DVDs, HD-DVD, (High Definition DVD), BLU-RAY, or other optical storage, magnetic cassettes, magnetic tape, magnetic disk storage, or other magnetic storage devices, or any other medium which can be used to store the desired information and which can be accessed by the architecture to store the desired information and which can be accessed by the architecture.

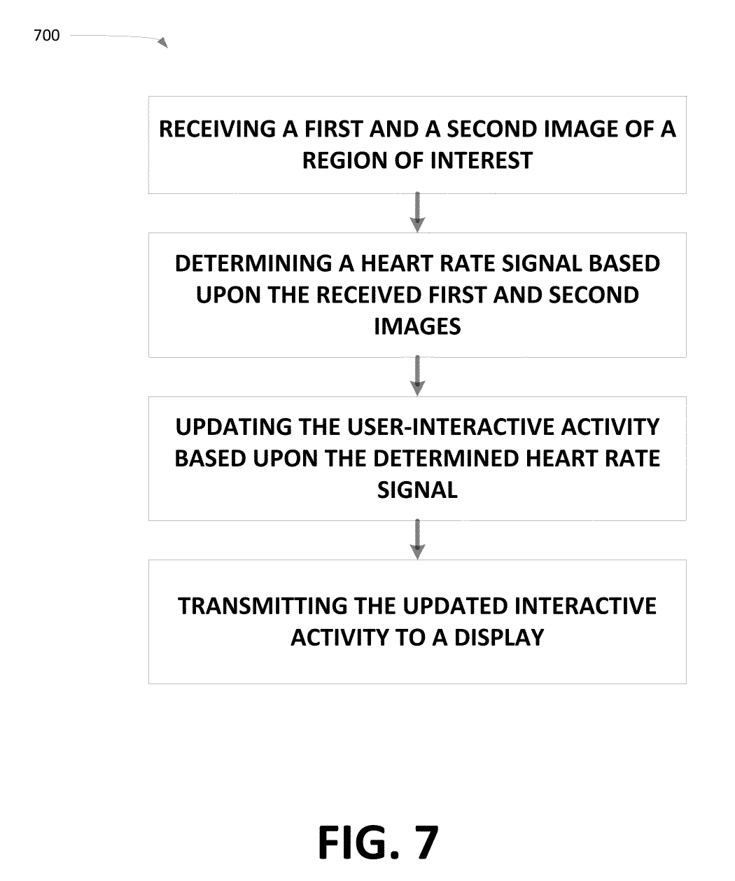

The memory may include one or more computer-executable instructions such as disclosed above in the call flows 600, 700 illustrated in FIGS. 6 and 7. The computer executable instructions may be executed by a processor, such as the processor disclosed above that is employed in devices such as capture devices and computing devices including gaming consoles. The processor is configured to perform the instructions of: (i) capturing a first image and a second image of a region of interest; (ii) aligning the second image with the first image in a first spectrum that is not sensitive to a characteristic, e.g., blood flow in a region of interest; (iii) applying a correction based upon the aligning step to the second image in a second spectrum that is sensitive to the characteristic; and (iv) measuring intensity of a signal of the second image in a second spectrum. According to another aspect, the processor in a capture device or computing device is configured to perform the instructions of (i) receiving a first and a second image of a region of interest; (ii) determining a heart rate signal based upon the received first and second images; and (iii) updating the user-interactive activity based upon the determined heart rate signal.

### Claims Section

A computer-implemented method is disclosed in an aspect of the application. The method includes a step of capturing a first image and a second image of a region of interest. The second image is aligned with the first image in a first spectrum that is sensitive to a characteristic to obtain a correction. Then, a correction based upon the above-mentioned alignment is applied to the second image in a second spectrum that is sensitive to the characteristic. In one aspect, the characteristic includes blood flow in the region of interest. In another aspect, the region of interest may include skin on a human body. In another aspect, the method may also include the step of measuring intensity of a computer-generated signal based on data received in the second spectrum for at least two captured image pairs that have undergone the aligning and applying steps. Moreover, the computer-generated signal may include a representation of heart rate. In another aspect, the first color channel may be selected from a red color channel, blue color channel, ultraviolet color channel and an infrared color channel. In another aspect the second color channel includes a green color channel.

Another aspect describes an image capture device. The device includes an image capture component that captures at least two successive image pairs of a region of interest. The devices also includes a memory that stores the at least two successive image pairs captured by the image capture component. The device also includes a processor, operably coupled to the memory and the image capture component. The processor is configured to perform the instructions of identifying a first spectrum that is not sensitive to a characteristic and a second spectrum that is sensitive to the characteristic for the at least two successive image pairs. The processor is also configured to perform the instructions of measuring intensity of a heart rate based upon a computer-generated signal based on data received in the second spectrum for the at least two successive image pairs. In one aspect the processor is further configured to perform the instructions of aligning a second image with a first image of one captured image pair in the first spectrum to obtain a correction. The processor is further configured to perform the instructions of applying the correction to the first or the second image in the second spectrum. In another aspect, the characteristic includes blood flow.

A computer-implemented system is described in even another aspect of the application. The system may include a means for capturing images. In one aspect, this may be an image capture device as described above. The system may also include a computing device operably connected to the image capture device. The system may also include a means for displaying the intensity of a heart rate signal. In one aspect this may be a display that is operably connected to the image capture device and the computing device for displaying the intensity of the heart rate signal. Further, the system may include a server operably connected to the image capture device and the computing device via a network.

A computer-implemented method for updating a user-interactive activity is described in even another aspect of the application. The method includes receiving at least two successive image pairs for a region of interest. The method also includes determining a heart rate signal based upon the received image pairs. The method further includes the step of updating the user-interactive activity based upon the determined heart rate signal. In one aspect, the method may include a means for transmitting the updated user-interactive activity to a display. In another aspect, the method may include a means for comparing a predefined input of a fitness goal of a user with the determined heart rate signal. In yet another aspect, the user-interactive activity may include a fitness module stored on a computing device. In yet another aspect, the determining step may include aligning the a first and a second image of each image pair in a first spectrum that is not sensitive to a characteristic to obtain a correction and applying the correction to the first or the second image in a second spectrum that is sensitive to the characteristic. In even a further aspect, the first spectrum is different from the second spectrum. In yet even a further aspect, the step of updating may include a means for notifying a server via a network of the determined heart rate, and receiving a supplemental user-interactive activity based upon the determined heart rate.

While the methods, systems and software applications have been described in terms of what are presently considered to be specific aspects, the disclosure need not be limited to the disclosed aspects. It is intended to cover various modifications and similar arrangements included within the spirit and scope of the claims, the scope of which should be accorded the broadest interpretation so as to encompass all such modifications and similar structures. The present application includes any and all aspects of the following claims.

What is claimed is:

1. A computer-implemented method comprising:
capturing a first image of a region of interest;
capturing a second image of the region of interest;
aligning the second image with the first image in a first spectrum that is not sensitive to a characteristic to obtain a correction; and
applying the correction to the first or the second image in a second spectrum that is sensitive to the characteristic.

2. The method of claim 1, wherein the characteristic includes blood flow in the region of interest.

3. The method of claim 1, wherein the region of interest includes skin on a human body.

4. The method of claim 1, further comprising:
measuring intensity of a computer-generated signal based on data received in the second spectrum for at least two captured image pairs that have undergone the aligning and applying steps.

5. The method of claim 4, wherein the computer-generated signal includes a representation of heart rate.

6. The method of claim 1, wherein
the first spectrum includes a first color channel selected from a red color channel, blue color channel, ultraviolet color channel and an infrared color channel.

7. The method of claim 1, wherein the second color channel includes a green color channel.

8. An image capture device comprising:
an image capture component that captures at least two successive image pairs of a region of interest;
a memory that stores the at least two successive image pairs captured by the image capture component; and
a processor, operably coupled to the memory and the image capture component, the processor configured to perform the instructions of:
identifying a first spectrum that is not sensitive to a characteristic and a second spectrum that is sensitive to the characteristic for the at least two successive image pairs, and
measuring intensity of a heart rate based upon a computer-generated signal based on data received in the second spectrum for the at least two successive image pairs.

9. The device of claim 8, wherein the processor is further configured to perform the instructions of:
aligning a second image with a first image of each captured image pair in the first spectrum to obtain a correction; and
applying the correction to the first or the second image in the second spectrum.

10. The device of claim 8, wherein the characteristic includes blood flow in the region of interest.

11. A computer-implemented system comprising:
the image capture device of claim 8; and
a computing device operably connected to the image capture device.

12. The system of claim 11, further comprising:
a display operably connected to the image capture device and the computing device for displaying the intensity of the heart rate signal.

13. The system of claim 11, further comprising:
a server operably connected to the image capture device and the computing device via a network.

14. A computer-implemented method for updating a user-interactive activity comprising:
receiving at least two successive image pairs for a region of interest;
determining a heart rate signal based upon the received image pairs; and
updating the user-interactive activity based upon the determined heart rate signal.

15. The method of claim 14, further comprising:
transmitting the updated user-interactive activity to a display.

16. The method of claim 14, further comprising:
comparing a predefined input of a fitness goal of a user with the determined heart rate signal.

17. The method of claim 14, wherein the user-interactive activity includes a fitness module stored on a computing device.

18. The method of claim 14, wherein the determining step includes:
aligning a first and a second image of each image pair in a first spectrum that is not sensitive to a characteristic to obtain a correction; and
applying the correction to the first or the second image in a second spectrum that is sensitive to the characteristic.

19. The method of claim 14, wherein the first spectrum is different from the second spectrum.

20. The method of claim 14, wherein the updating step includes notifying a server via a network of the determined heart rate, and receiving a supplemental user-interactive activity based upon the determined heart rate.
